(19) United States
(12) Patent Application Publication
(10) Pub. No.: US 2011/0288357 A1
(43) Pub. Date: Nov. 24, 2011

(54) PROCESS FOR THE PREPARATION OF AN OLEFINIC PRODUCT

(76) Inventors: Leslie Andrew Chewter, Amsterdam (NL); Jeroen Van Westrenen, Amsterdam (NL); Ferry Winter, Amsterdam (NL)

(21) Appl. No.: 13/140,885

(22) PCT Filed: Dec. 21, 2009
(86) PCT No.: PCT/EP2009/067628

§ 371 Date: Jul. 11, 2011

(30) Foreign Application Priority Data
Dec. 22, 2008 (EP) 08172574.9

(51) Int. Cl. C07C 1/20

(52) U.S. Cl. 585/638

(57) ABSTRACT

A process for the preparation of an olefinic product, the process comprising reacting an oxygenate feedstock in a reactor in the presence of catalyst comprising an aluminosilicate having one-dimensional 10 membered ring channels, to produce the olefinic product, wherein before reacting the oxygenate feedstock to produce the olefinic product, the acidity of the aluminosilicate has been increased by at least 5% by treating the aluminosilicate with an aqueous solution comprising at least one of a chelating agent and an acid, wherein the increase in acidity is based on the acidity of the aluminosilicate before treatment.

### FIELD OF THE INVENTION

This invention relates to a process for the preparation of an olefinic product, in particular including lower olefins, more in particular ethylene and/or propylene. In one aspect this invention relates to a process for the conversion of oxygenates into olefins.

### BACKGROUND OF THE INVENTION

Processes for the preparation of olefins from oxygenates are known in the art. Of particular interest is often the production of light olefins, in particular ethylene and/or propylene. The oxygenate feedstock can for example comprise methanol and/or dimethylether, and an interesting route includes their production from synthesis gas derived from e.g. natural gas or via coal gasification.

For example, WO2007/135052 discloses a process wherein an alcohol and/or ether containing oxygenate feedstock and an olefinic co-feed are reacted in the presence of zeolite having one-dimensional 10-membered ring channels to prepare an olefinic reaction mixture, and wherein part of the obtained olefinic reaction mixture is recycled as olefinic co-feed. With a methanol and/or dimethylether containing feedstock, and an olefinic co-feed comprising C4 and/or C5 olefins, an olefinic product rich in light olefins can be obtained.

### SUMMARY OF THE INVENTION

It has surprisingly been found by the inventors of the present invention that treatment of an aluminosilicate in an oxygenate to olefin process by an acid or chelating agent or a combination thereof can increase the activity of the catalyst during the subsequent process of converting oxygenates to olefins.

According to a first aspect of the invention, there is provided a process for the preparation of an olefinic product, the process comprising reacting an oxygenate feedstock in a reactor in the presence of catalyst comprising an aluminosilicate having one-dimensional 10 membered ring channels, to produce the olefinic product, wherein before reacting the oxygenate feedstock to produce the olefinic product, the acidity of the aluminosilicate has been increased by at least 5% by treating the aluminosilicate with an aqueous solution comprising at least one of a chelating agent and an acid, wherein the increase in acidity is based on the acidity of the aluminosilicate before treatment.

Thus, the invention provides a process for the preparation of an olefinic product, the process comprising treating catalyst comprising an aluminosilicate having one-dimensional 10-membered ring channels with at least one of a chelating agent and an acid to remove a portion of the alumina; reacting an oxygenate feedstock in a reactor in the presence of said treated aluminosilicate, to produce an olefinic product.

The process according to the invention typically results in removal of a portion of alumina which would normally be expected to reduce the acidity and therefore the activity of the catalyst. However, against such expectations, it has been found that the removal of alumina, before the process for preparing an olefin, actually increases the activity of the catalyst.

Without wishing to be bound by any particular hypothesis or theory, it is considered that a portion of non-framework alumina, sometimes referred to as extra framework alumina, is removed. This is especially useful for aluminosilicate with one-dimensional 10-membered ring channels as it provides for increased access to the one-dimensional channels which in turn increases the acidity of the aluminosilicate.

The acidity of the aluminosilicate which is increased is typically the “accessible acidity”, that is the effective acidity of the aluminosilicate which may be measured by a techniques that rely on the access of a probing agent to acid sites. References hereinafter to acidity thus refer to accessible acidity, unless otherwise provided. Blocked access such as by non-framework alumina is thought to reduce the measured “accessible acidity” with respect to a theoretical acidity, and by removing non-framework alumina the accessible acidity can increase.

### DETAILED DESCRIPTION OF THE INVENTION

Typically the aluminosilicate has been treated with at least one of a chelating agent and an acid, such as sulphuric acid and hydrochloric acid, and especially those comprising one, two or more carboxylic acid groups such as citric acid and acetic acid.

Preferred acids include one or more of: oxalic acid, tartaric acid and nitric acid with oxalic acid especially preferred. The acid may be a multi-protic acid i.e. the acid molecule may have two or more protons.

Where mono-protic acids are used, preferably these are diluted to the preferred pH.

The chelating agent may be at least one of ethylenediaminetetraacetic acid (EDTA), nitrilo tri acetic acid (NTA), tri-phosphates such as sodium tri-phosphates (STP); tri-phosphoric acid, polyphosphates and tri- or poly-metaphosphate. Some of these chelating agents, such as EDTA, are also acids. For some embodiments, the acid groups may have been exchanged with another ion, such as Na, in place of their protons. Normally however the acids have at least one proton present on each molecule.

Preferably the solution of the at least one of a chelating agent and acid has a pH of less than 5 preferably less than 3; preferably the pH is more than 0.5. The pH is measured at 25° C. The pH of the solution can be adjusted by adding a mono-protic acid such as nitric acid.

Normally the treatment involves the step of contacting the aluminosilicate with an aqueous acid solution, the aqueous acid solution preferably having a molarity in the range of 0.01-3M, more preferably 0.05-2, especially 0.075-1.25M. Normally the treatment involves the step of contacting the aluminosilicate with an aqueous acid solution, the aqueous acid solution preferably being from room temperature to 200° C., preferably 60-100° C. during contact.

The treatment is typically performed for 0.2-24 hours, preferably 0.5-5 hours, especially around 1 hour.

The longer the time, and higher the molarity and temperature and the lower the pH, the more severe the process will be and therefore the more effective the process will be at removing a portion of alumina. Therefore these parameters can be offset from one another, for example, a short treatment time can be effective if the molarity and/or temperature is increased and/or the pH decreased to compensate for the short treatment time.

Treatment of the aluminosilicate in accordance with the invention has been found to increase the acidity, however as the severity of the process is increased (such as by longer treatment time or higher temperature) the resulting acidity will reach a maximum and then start to decrease. Extensive treatment, after the maximum acidity, would eventually result in nearly unreactive (inactive) silica. Therefore it is preferred to treat the aluminosilicate by a certain severity, as detailed herein, such that it increases its acidity compared to untreated aluminosilicate, and ideally, such that it reaches its maximum acidity. It is believed that the increase is mainly due to the removal of non-framework alumina from the micropores of the aluminosilicate, and the decrease is mainly due to a removal of framework alumina.

After treatment with said aqueous solution, the acidity of the aluminosilicate is typically more than 5% greater, preferably more than 10% greater than the acidity before treatment with the said aqueous solution. Reference herein to the acidity is to the acidity determined by n-propylamine adsorption.

After treatment with said aqueous solution, the micropore volume of the aluminosilicate is typically more than 5% greater, preferably more than 10% greater, than the micropore volume before treatment with the said aqueous solution.

After treatment with said aqueous solution, the overall surface area of the aluminosilicate is typically more than 5% greater, preferably more than 10% greater, than the overall surface area before treatment with the said aqueous solution.

The absolute values of acidity, micropore volume, and/or overall surface area depend on various factors such as the characteristics of the aluminosilicate.

As a result of the treatment with said aqueous solution, the measured silica-to-alumina-ratio (SAR) typically increases.

The process is carried out in presence of an aluminosilicate having one-dimensional 10-membered ring channels. These are understood to be aluminosilicates having only 10-membered ring channels in one direction which are not intersected by other 8, 10 or 12-membered ring channels from another direction.

Preferably, the aluminosilicate is selected from the group of TON-type (for example zeolite ZSM-22), MTT-type (for example zeolite ZSM-23), STF-type (for example SSZ-35), SFF-type (for example SSZ-44), EUO-type (for example ZSM-50), and EU-2-type aluminosilicates or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type aluminosilicates are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type aluminosilicates are more particularly described in e.g. U.S. Pat. No. 4,397,827. For purposes of the present invention, EU-2 is considered to include its isotypes, e.g., ZSM-48.

In a further preferred embodiment an aluminosilicate of the MTT-type, such as ZSM-23, and/or a TON-type, such as ZSM-22 is used.

The aluminosilicate is typically a molecular sieve. An aluminosilicate molecular sieve is also referred to as a zeolite. Molecular sieve and zeolite types are for example defined in Ch. Baerlocher and L. B. McCusker, Database of Zeolite Structures: http://www.iza-structure.org/databases/, which database was designed and implemented on behalf of the Structure Commission of the International Zeolite Association (IZA-SC), and based on the data of the 4th edition of the Atlas of Zeolite Structure Types (W. M. Meier, D. H. Olson and Ch. Baerlocher).

Preferably, aluminosilicates in the hydrogen form are used in the reactor to produce the olefinic product, e.g., HZSM-22, HZSM-23, and HZSM-48, HZSM-5. Preferably at least 50% w/w, more preferably at least 90% w/w, still more preferably at least 95% w/w and most preferably 100% of the total amount of aluminosilicate used is in the hydrogen form. When the aluminosilicates are prepared in the presence of organic cations the aluminosilicate may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 300° C. The aluminosilicates obtained after ion-exchange are also referred to as being in the ammonium form.

Normally treatment with said aqueous solution is performed on the aluminosilicate when it is in its ammonium form although for certain embodiments treatment could also be performed on the aluminosilicate in another form such as the sodium or potassium form. Furthermore, the treatment could also be performed after the aluminosilicate is obtained in the hydrogen form, or the treatment could even be conducted after steaming the aluminosilicate containing catalyst in the hydrogen form at elevated temperatures, or yet in another way.

The silica-to-alumina-ratio (SAR) is defined as the molar ratio of SiO₂/Al₂O₃ corresponding to the composition of the aluminosilicate. Normally the zeolite has a SAR in the range of from 1 to 500, particularly a SAR in the range of from 10 to 200. Where the aluminosilicate comprises a ZSM23 zeolite, preferably the SAR is in the range of 2-120, more preferably 10-100, especially 30-80. Good performance in terms of activity and selectivity of the conversion of oxygenate to lower olefins has been observed at a SAR of about 50. Where the aluminosilicate comprises a ZSM22 zeolite preferably the SAR is in the range of 40-170, more preferably 70-140, especially 90-110. Good performance in terms of activity and selectivity of the conversion of oxygenate to lower olefins has been observed at a SAR of about 100.

It is desirable to provide a catalyst having good mechanical or crush strength, or attrition resistance, because in an industrial environment the catalyst is often subjected to rough handling, which tends to break down the catalyst into powder-like material. The latter causes problems in the processing. Preferably the zeolite is therefore mixed with a matrix and a binder material and then spray dried or shaped to the desired shape, such as pellets. Examples of suitable binder materials include active and inactive materials and synthetic or naturally occurring zeolites as well as inorganic materials such as clays, silica, alumina, silica-alumina, titania, zirconia and aluminosilicate. For present purposes, inert materials, such as silica, are preferred because they may prevent unwanted side reactions which may take place in case a more acidic material, such as alumina or silica-alumina is used. Preferably the catalyst used in the process of the present invention comprises, in addition to the aluminosilicate, 2 to 90 wt %, preferably 10 to 85 wt % of a binder material.

Preferably the treatment according to the invention is performed before the addition of such binder/matrix materials.

In a special embodiment the reaction is performed in the presence of a more-dimensional molecular sieve, such as ZSM-5. Suitably to this end the oxygenate conversion catalyst comprises at least 1 wt %, based on total molecular sieve in the oxygenate conversion catalyst, of a further molecular sieve having more-dimensional channels, in particular at least 5 wt %, more in particular at least 8 wt %.

The further molecular sieve having more-dimensional channels is understood to have intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. The second molecular sieve can be for example a FER type zeolite which is a two-dimensional structure and has 8- and 10-membered rings intersecting each other. Preferably however the intersecting channels in the second molecular sieve are each 10-membered ring channels. Thus the second molecular sieve may be a zeolite, or a SAPO-type (silicoaluminophosphate) molecular sieve. More preferably however the second molecular sieve is a zeolite. A preferred second molecular sieve is an MFI-type zeolite, in particular zeolite ZSM-5.

The presence of the further molecular sieve in the oxygenate conversion catalyst was found to improve stability (slower deactivation during extended runs) and hydrothermal stability compared to a catalyst with only the one-dimensional molecular sieve and without the more-dimensional molecular sieve. Without wishing to be bound by a particular hypothesis or theory, it is presently believed that this is due to the possibility for converting larger molecules by the second molecular sieve having more-dimensional channels, that were produced by the first molecular sieve having one-dimensional 10-membered ring channels, and which would otherwise form coke. When the one-dimensional aluminosiclicate and the more-dimensional molecular sieve are formulated such that they are present in the same catalyst particle, such as in a spray-dried particle, this intimate mix was found to improve the selectivity towards ethylene and propylene, more in particular towards ethylene.

The weight ratio between the aluminosilicate having one-dimensional 10-membered ring channels, and the further molecular sieve having more-dimensional channels can be in the range of from 1:100 to 100:1. Preferably the further molecular sieve is the minority component, i.e. the above weight ratio is 1:1 to 100:1, more preferably in the range of 9:1 to 2:1.

Preferably the further molecular sieve is an MFI-type aluminosilicate, in particular zeolite ZSM-5, having a Silica-to-Alumina ratio SAR of at least 60, more preferably at least 80, even more preferably at least 100, yet more preferably at least 150. At higher SAR the percentage of C4 saturates in the C4 totals produced is minimized. In special embodiments the oxygenate conversion catalyst can comprise less than 35 wt % of the further molecular sieve, based on the total molecular sieve in the oxygenate conversion catalyst, in particular less than 20 wt %, more in particular less than 18 wt %, still more in particular less than 15 wt %.

In one embodiment the oxygenate conversion catalyst can comprise more than 50 wt %, at least 65 wt %, based on total molecular sieve in the oxygenate conversion catalyst, of the aluminosilicate having one-dimensional 10-membered ring channels. The presence of a majority of such aluminosilicate strongly determines the predominant reaction pathway.

The more-dimensional molecular sieve may be subjected to the treatment with an aqueous solution according to the present invention, although this is not essential.

Typically the oxygenate conversion catalyst deactivates in the course of the process. Conventional catalyst regeneration techniques can be employed, such as burning of coke in a regenerator. The aluminosilicate having one-dimensional 10 membered ring channels used in the process of the present invention can have any shape known to the skilled person to be suitable for this purpose, for it can be present in the form of spray-dried particles, spheres, tablets, rings, extrudates, etc. Extruded catalysts can be applied in various shapes, such as, cylinders and trilobes. If desired, spent oxygenate conversion catalyst can be regenerated and recycled to the process of the invention.

Spherical particles are normally obtained by spray drying. Preferably the spray dried particles are smaller than 300 micron, preferably less than 200 micron.

Extruded catalysts can be applied in various shapes, such as, cylinders and trilobes. If desired, spent zeolite can be regenerated and recycled to the process of the invention.

Preferably the oxygenate feedstock is reacted to produce the olefinic product in the presence of an olefinic co-feed, although for certain embodiments an olefinic co-feed is not necessary. By an olefinic composition or stream, such as an olefinic product, product fraction, fraction, effluent, reaction effluent or the like is understood a composition or stream comprising one or more olefins, unless specifically indicated otherwise. Other species can be present as well. The olefinic composition or stream can comprise one type of olefin or a mixture of olefins.

By an olefin is understood an organic compound containing at least two carbon atoms connected by a double bond.

In particular the olefinic co-feed, where present, can contain a mixture of olefins. Apart from olefins, the olefinic co-feed may contain other hydrocarbon compounds, such as for example paraffinic compounds. Preferably the olefinic co-feed comprises an olefinic portion of more than 50 wt %, more preferably more than 60 wt %, still more preferably more than 70 wt %, which olefinic portion consists of olefin(s). The olefinic co-feed, where present, can also consist essentially of olefin(s).

Any non-olefinic compounds in the olefinic co-feed are preferably paraffinic compounds. Such paraffinic compounds are preferably present in an amount in the range from 0 to 50 wt %, more preferably in the range from 0 to 40 wt %, still more preferably in the range from 0 to 30 wt %.

The olefin can be a mono-olefin, having one double bond, or a poly-olefin, having two or more double bonds. Preferably olefins present in the olefinic co-feed, where present, are mono-olefins. C4 olefins, also referred to as butenes (1-butene, 2-butene, iso-butene, and/or butadiene), in particular C4 mono-olefins, are preferred components in the olefinic co-feed.

Preferably the olefinic co-feed is at least partially obtained by a recycle stream formed by recycling a suitable fraction of the reaction product comprising C4 olefin.

The skilled artisan knows how to obtain such a fractions from the olefinic reaction effluent such as by distillation.

In one embodiment at least 70 wt % of the olefinic co-feed, during normal operation, is formed by the recycle stream, preferably at least 90 wt %, more preferably at least 99 wt %. Most preferably the olefinic co-feed is during normal operation formed by the recycle stream, so that the process converts oxygenate feedstock to predominantly light olefins without the need for an external olefins stream. During normal operation means for example in the course of a continuous operation of the process, for at least 70% of the time on stream. The olefinic co-feed may need to be obtained from an external source, such as from a catalytic cracking unit or from a naphtha cracker, during start-up of the process, when the reaction effluent comprises no or insufficient C4+ olefins.

A particularly preferred olefinic recycle stream is a C4 fraction containing C4 olefin(s), but which can also contain a significant amount of other C4 hydrocarbon species, in particular C4 paraffins, because it is difficult to economically separate C4 olefins and paraffins, such as by distillation.

In a preferred embodiment the olefinic co-feed and preferably also the recycle stream comprises C4 olefins and less than 10 wt % of C5+ hydrocarbon species, more preferably at least 50 wt % of C4 olefins, and at least a total of 70 wt % of C4 hydrocarbon species.

The olefinic co-feed and preferably also the recycle stream, can in particular contain at least a total of 90 wt % of C4 hydrocarbon species. In a preferred embodiment, the olefinic co-feed comprises less than 5 wt % of C5+ olefins, preferably less than 2 wt % of C5+ olefins, even more preferably less than 1 wt % of C5+ olefins, and likewise the recycle stream. In another preferred embodiment, the olefinic co-feed, comprises less than 5 wt % of C5+ hydrocarbon species, preferably less than 2 wt % of C5+ hydrocarbon species even more preferably less than 1 wt % of C5+ hydrocarbon species, and likewise the recycle stream. Preferably the olefinic portion of the olefinic co-feed, and of the recycle stream, comprises at least 90 wt % of C4 olefins, more preferably at least 99 wt %. Butenes as co-feed have been found to be particularly beneficial for high ethylene selectivity.

One particularly suitable recycle stream consists essentially, i.e. for at least 99 wt %, of 1-butene, 2-butene (cis and trans), isobutene, n-butane, isobutene, butadiene.

In certain embodiments, the recycle stream can also comprise propylene. This may be preferred when a particularly high production of ethylene is desired, so that part or all of the propylene produced, such as at least 5 wt % thereof, is recycled together with C4 olefins.

The oxygenate feedstock comprises oxygenate species having an oxygen-bonded methyl group, such as methanol, dimethylether. Preferably the oxygenate feedstock comprises at least 50 wt % of methanol and/or dimethylether, more preferably at least 80 wt %, most preferably at least 90 wt %.

The oxygenate feedstock can be obtained from a different or separate reactor, which converts methanol at least partially into dimethylether and water. Water may be removed by e.g. distillation. In this way, less water is present in the process of converting oxygenate to olefins, which has advantages for the process design and lowers the severity of hydrothermal conditions the catalyst is exposed to. The oxygenate feedstock can comprise an amount of water, preferably less than 10 wt %, more preferably less than 5 wt %. Preferably the oxygenate feedstock contains essentially no hydrocarbons other than oxygenates, i.e. less than 5 wt %, preferably less than 1 wt %.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 10:1 to 1:10, more preferably in the range of 5:1 to 1:5 and still more preferably in the range of 3:1 to 1:3.

In a preferred embodiment wherein the oxygenate comprises only one oxygen-bonded methyl group, such as methanol, the molar ratio preferably lies in the range from 5:1 to 1:5 and more preferably in the range of 2.5:1 to 1:2.5.

In another preferred embodiment wherein the oxygenate comprises two oxygen-bonded methyl groups, such as for example dimethylether, the molar ratio preferably lies in the range from 5:2 to 1:10 and more preferably in the range of 2:1 to 1:4. Most preferably the molar ratio in such a case is in the range of 1.5:1 to 1:3.

The process can be carried out over a wide range of temperatures and pressures. Suitably, however, the oxygenate feed and olefinic co-feed, where present, are contacted with the aluminosilicate at a temperature in the range from 200° C. to 650° C. In a further preferred embodiment the temperature is in the range from 250° C. to 600° C., more preferably in the range from 300° C. to 550° C., most preferably in the range from 450° C. to 550° C. Preferably the reaction to produce an olefinic product is conducted at a temperature of more than 450° C., preferably at a temperature of 460° C. or higher, more preferably at a temperature of 490° C. or higher. At higher temperatures a higher activity and ethylene selectivity is observed. Aluminosilicates having one-dimensional 10-membered ring channels can be operated under oxygenate conversion conditions at such high temperatures with acceptable deactivation due to coking, contrary to aluminosilicates with smaller pores or channels, such as 8-membered ring channels. Temperatures referred to hereinabove represent reaction temperatures, and it will be understood that a reaction temperature can be an average of temperatures of various feed streams and the catalyst in the reaction zone.

In addition to the oxygenate, and the olefinic co-feed (when present), a diluent may be fed into the reactor system. It is preferred to operate without a diluent, or with a minimum amount of diluent, such as less than 200 wt % of diluent based on the total amount of oxygenate feed, in particular less than 100 wt %, more in particular less than 20 wt %. Any diluent known by the skilled person to be suitable for such purpose can be used. Such diluent can for example be a paraffinic compound or mixture of compounds. Preferably, however, the diluent is an inert gas. The diluent can be argon, nitrogen, and/or steam. Of these, steam is the most preferred diluent. For example, the oxygenate feed and optionally olefinic co-feed can be diluted with steam, for example in the range from 0.01 to 10 kg steam per kg oxygenate feed.

In one embodiment small amounts of water are added during the reaction to produce the olefinic product in order to improve the stability of the catalyst by reducing coke formation.

Typically the olefinic product or reaction effluent is fractionated. The skilled artisan knows how to separate a mixture of hydrocarbons into various fractions, and how to work up fractions further for desired properties and composition for further use. The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation. It is within the skill of the artisan to determine the correct conditions in a fractionation column to arrive at such a separation. He may choose the correct conditions based on, inter alia, fractionation temperature, pressure, trays, reflux and reboiler ratios.

In one embodiment, a light olefinic fraction comprising ethylene and a heavier olefinic fraction comprising C4 olefins and less than 10 wt % of C5+ hydrocarbon species can be obtained. Preferably also a water-rich fraction is obtained. Also a lighter fraction comprising methane, carbon monoxide, and/or carbon dioxide can be obtained, as well as one or more heavy fractions comprising C5+ hydrocarbons. Such heavy fraction can for example be used as gasoline blending component.

In the process also a significant amount of propylene is produced. The propylene can form part of the light olefinic fraction comprising ethene, and which can suitably be further fractionated into various product components. Propylene can also form part of the heavier olefinic fraction comprising C4 olefins. The various fractions and streams referred to herein, in particular the recycle stream, can be obtained by fractionating in various stages, and also by blending streams obtained during the fractionation. Typically, an ethylene and a propylene stream of predetermined purity such as pipeline grade, polymer grade, chemical grade or export quality will be obtained from the process, and also a stream rich in C4 comprising C4 olefins and optionally C4 paraffins. It shall be clear that the heavier olefinic fraction comprising C4 olefins, forming the recycle stream, can be composed from quantities of various fractionation streams. So, for example, some amount of a propylene-rich stream can be blended into a C4 olefin-rich stream. In a particular embodiment at least 90 wt % of the heavier olefinic fraction comprising C4 olefins can be formed by the overhead stream from a debutaniser column receiving the bottom stream from a depropanizer column at their inlet, more in particular at least 99 wt % or substantially all.

Suitably the olefinic reaction effluent comprises less than 10 wt %, preferably less than 5 wt %, more preferably less than 1 wt %, of C6-C8 aromatics. Producing low amounts of aromatics is desired since any production of aromatics consumes oxygenate which is therefore not converted to lower olefins.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed. Such olefins may for example be obtained from a steam cracker, a catalytic cracker, alkane dehydrogenation (e.g. propane or butane dehydrogenation). Further, such olefins can be bought from the market.

In a special embodiment the olefins for such start-up are obtained from a previous process that converted oxygenates, with or without olefinic co-feed, to olefins. Such a previous process may have been located at a different location or it may have been carried out at an earlier point in time.

When a molecular sieve with more-dimensional channels such as ZSM-5 is present in the oxygenate conversion catalyst, even in minority compared to the aluminosilicate with one-dimensional ring channels, start up is possible without an olefinic co-feed from an external source. ZSM-5 for example is able to convert an oxygenate to an olefin-containing product, so that a recycle can be established.

The reactor system used may be any reactor known to the skilled person and may for example contain a fixed bed, moving bed, fluidized bed, riser reactor and the like. A riser reactor system is preferred, and in a particular embodiment a riser reactor system comprising a plurality of serially arranged riser reactor stages is used.

### EXAMPLES

Embodiments of the invention will now be described, by way of example only.

An MTT zeolite (ZSM-23) with a silica-to-alumina ratio of 46 supplied by Zeolyst International in an ammonium (NH₄⁺) form was subjected to de-alumination treatment in aqueous acid solutions in accordance with the present invention.

Various samples of dry zeolite powder were suspended in an aqueous solution containing (i) oxalic acid in 0.1 M (ii) tartaric acid in 0.1 M and (iii) 0.1 M nitric acid.

Typically, 40 grams of zeolite was used in 400 ml solution at 80° C. and stirred for 1 h, after which the suspension was allowed to cool to room temperature. After the treatment the samples were filtered, washed and subsequently dried at 120° C.

In all samples a single de-alumination step was conducted. Additionally for oxalic and tartaric acid treatments, separate samples were de-aluminated twice.

The catalytic performance of the treated samples was determined in conversion experiments of a 1-butene/methanol feed.

Prior to testing, a sample of the treated zeolite powder was pressed into tablets and the tablets were broken into pieces and sieved. For catalytic testing, the sieve fraction of 60-80 mesh has been used. Prior to test performance, the fresh catalyst was treated ex-situ in air at 600° C. for 2 hours.

In these examples methanol and 1-butene were used for conversion experiments at 525° C., at a Gas hourly space velocity (GHSV) of 29,000 ml·g⁻¹·h⁻¹, 3 vol % 1-butene and 6 vol % methanol balanced in N₂. The catalyst was heated in N₂ to the reaction temperature and a mixture consisting of methanol, 1-butene balanced in N₂ was passed over the catalyst at atmospheric pressure (1 bar). Gas hourly space velocity is based on total gas flow (ml·g⁻¹·h⁻¹).

The effluent from the reactor was analyzed by gas chromatography (GC) to determine the product composition. The composition has been calculated on a weight basis of all hydrocarbons analyzed. The selectivity has been defined by the division of the mass of product by the sum of the masses of all products.

Table 1 below shows a performance evaluation of ZSM-23, untreated and after acid leaching or de-alumination steps with oxalic acid, tartaric acid and nitric acid.

TABLE 1

| | Untreated | Oxalic | Tartaric | Nitric |
|---|---|---|---|---|
| Catalyst | (com- | acid | acid | acid |
| Sample | parative) | treated | treated | treated |
| Time (h) | 5.4 | 5.4 | 5.4 | 5.4 |
| C2⁼/C3⁼ wt- | 0.24 | 0.32 | 0.28 | 0.30 |
| ratio | | | | |
| C2⁼-C3⁼ yield | 50.2 | 63.2 | 59.9 | 60.7 |
| wt % ⁽ⁱ⁾ | | | | |
| C2-C4 yield | 71.9 | 85.4 | 81.8 | 82.6 |
| wt % ⁽ⁱ⁾ | | | | |
| C5-C8 yield | 28.1 | 14.3 | 17.7 | 17.4 |
| wt % ⁽ⁱ⁾ | | | | |
| DME | 9.5 | >15.8 | 13.7 | 13.7 |
| breakthrough | | | | |
| ⁽ⁱⁱ⁾ (h) | | | | |

Oxalic acid treatment
⁽ⁱ⁾ wt % of total HC measured in product stream;
⁽ⁱⁱ⁾ time at which DME is present in product stream.

From Table 1 it can be observed that the acid de-alumination treatments increases the desired C2-C4 yields significantly. The best result was observed with an oxalic acid treatment (85.4% compared to 71.9%). Moreover the time before which the reactant intermediate dimethylether (DME) is present in the product (“DME breakthrough” representing a loss of catalyst stability) is much longer—more than 15.8 hours compared to 9.5 hours for the untreated sample which indicates that the catalyst retains more activity after prolonged time on stream i.e. a higher stability.

These results are particularly surprising because the activity and selectivity of the resulting molecular sieve are both improved—the skilled person would normally expect a “pay-off” between selectivity (shown by a higher C2-C4 yield) and activity (shown by the DME breakthrough). That is, improvement in one of selectivity or activity would be expected to result in a deterioration in the other of selectivity and activity.

In a separate experiment the oxalic acid treatment was performed twice, which produced a C2-C4 yield, after 5.4 hours on stream, of 81.5%, compared to 71.9% for the untreated sample. However the twice de-aluminated sample had a slightly smaller C2-C4 yield than the single de-aluminated sample, caused, it is thought, by the loss of framework as well as non-framework alumina. However, the twice de-aluminated sample retained more activity with increasing reaction time as represented by the delayed DME breakthrough after 15.8 hours compared to 9.5 hours for the untreated sample.

Furthermore, from Table 1 it can be observed that treatment with an aqueous solution containing tartaric acid or nitric acid also increases the desired C2-C4 yield compared to the untreated sample. However, the treated samples with nitric acid or tartaric acid had a slightly smaller C2-C4 yield than the oxalic acid sample treated once.

Various experiments were also conducted to characterize several parameters the catalyst before and after the oxalic acid treatment, and the results are shown below in Table 2.

TABLE 2
Sample
charac

[symbol]

| | | Oxalic |
|---|---|---|
| | Untreated | acid |
| | ZSM-23 | treated |
| Surface Area | 188 | 217 |
| m²/g | | |
| Micropore | 0.050 | 0.061 |
| volume ml/g | | |
| Acidity | 0.27 | 0.34 |
| mmol/g | | |

[symbol]

indicates data missing or illegible when filed

In separate experiments tartaric acid and nitric acid were used to treat the zeolite. The acidity of the zeolite was found to increase to 0.36 and 0.37 compared to 0.27 mmol/g for the untreated ZSM-23. For these characterisation experiments, the surface area and micropore volume were measured by N2 physisorption, and the acidity by n-propylamine adsorption. Various techniques may be used to determine surface area and micropore volume values and different absolute values may be found by using different techniques for the same parameter. However the different techniques will show the same relative effects in surface area micropore volume (see IUPAC: http://www.iupac.org/publications/pac/1976/pdf/4601×0071.pdf), and acidity.

The acidity determination method used was a thermogravimetric method based on work done by Professor Raymond Gorte of the University of Pennsylvania. In this method, the sample is heated to 500° C. in a flowing inert gas in a thermogravic analyzer (TGA). The temperature is reduced to 100° C. and the desired base (n-propylamine) is allowed to flow over the sample and be absorbed by the acid sites. After a set time the sample is heated to some intermediate temperature, typically 230° C., to desorb any physisorbed base and then ramped to 500° C. The acidity is calculated by dividing the weight loss during the final ramp by the weight of the sample.

These results clearly show the increased acidity and activity of the examples treated with oxalic acid according to one embodiment of the present invention.

Thus it is shown from the results above that de-alumination of a catalyst comprising a molecular sieve having one-dimensional 10-membered ring channels prior to conducting a methanol to olefin conversion can have improved stability on stream and improved activity.

1. A process for the preparation of an olefinic product, the process comprising reacting an oxygenate feedstock in a reactor in the presence of catalyst comprising an aluminosilicate having one-dimensional 10 membered ring channels, to produce the olefinic product, wherein before reacting the oxygenate feedstock to produce the olefinic product, the acidity of the aluminosilicate has been increased by at least 5% by treating the aluminosilicate with an aqueous solution comprising at least one of a chelating agent and an acid, wherein the increase in acidity is based on the acidity of the aluminosilicate before treatment.

2. A process as claimed in claim 1, wherein the aluminosilicate comprises non-framework alumina and at least a portion of the non-framework alumina is removed by the treatment with the aqueous solution.

3. A process according to claim 1, wherein the aluminosilicate has been treated with an acid.

4. A process as claimed in claim 1, wherein the acid comprises a carboxylic acid group.

5. A process as claimed in claim 1, wherein the acid is oxalic acid or tartaric acid.

6. A process as claimed in claim 1, wherein the solution comprises at least one compound selected from the group consisting of ethylenediaminetetraacetic acid, nitrilo tri acetic acid, tri-phosphates; tri-phosphoric acid, polyphosphates and tri- or poly-metaphosphate.

7. A process as claimed in claim 1, wherein a solution of the at least one of a chelating agent and acid has a pH of less than 5.

8. A process as claimed in claim 1, wherein the treatment involves the step of contacting the aluminosilicate with an aqueous acid solution, the aqueous acid solution having a molarity in the range of 0.01-3M.

9. A process as claimed in claim 1, wherein the aluminosilicate has been combined with a binder following treatment with said aqueous solution and before reacting the oxygenate feedstock to produce the olefinic product.

10. A process as claimed in claim 1, wherein after treatment with said aqueous solution, the micropore volume of the aluminosilicate is more than 5% greater, than the micropore volume before treatment with the aqueous solution.

11. A process as claimed in claim 1, wherein after treatment with said aqueous solution, the acidity of the aluminosilicate is more than 10% greater than the acidity before treatment with said aqueous solution.

12. A process as claimed in claim 1, wherein the aluminosilicate comprises at least one of a TON-type aluminosilicate, and an MTT-type aluminosilicate.

13. A process as claimed in claim 1, wherein the molecular sieve comprises an MTT type aluminosilicate which after treatment with said aqueous solution has a silica to alumina ratio in the range of 2-200.

14. A process as claimed in claim 1, wherein the reaction is performed in the presence of a more-dimensional molecular sieve.

15. A process as claimed in claim 1, wherein the oxygenate feedstock is reacted to produce the olefinic product in the presence of an olefinic co-feed.
